(19) United States
(12) Patent Application Publication
(10) Pub. No.: US 2014/0045254 A1
(43) Pub. Date: Feb. 13, 2014

(54) CELL SELF-ASSEMBLY ARRAY CHIP AND MANUFACTURING METHOD THEREOF

(71) Applicant: NATIONAL TSING HUA UNIVERSITY, Hisnchu (TW)

(72) Inventors: Fan-Gang TSENG, Hsinchu (TW); Tsung-Ju CHEN, Hsinchu (TW); Yu-Cheng CHANG, Hsinchu (TW)

(73) Assignee: NATIONAL TSING HUA UNIVERSITY, Hisnchu (TW)

(21) Appl. No.: 13/723,969

(22) Filed: Dec. 21, 2012

(30) Foreign Application Priority Data
Aug. 10, 2012 (TW) 101128953

(51) Int. Cl. C12M 1/12

(52) U.S. Cl. CPC C12M 25/06
USPC 435/289.1; 29/428; 29/525.01; 29/458

(57) ABSTRACT

A cell self-assembly array chip comprises a first substrate, a second substrate and a plurality of spacer elements. The first substrate, the spacer elements and the second substrate are stacked to be one in turn, wherein the spacer elements are annularly arranged at interval and disposed on the second substrate, so as to define a cell self-assembly region and at least one drawing channel. Furthermore, because the liquid portion of cell suspension horizontally and radially passed the drawing channel outward and the radially outward pulling force generated by the evaporation effect of the liquid portion, so as to cause the cells are arranged by the cell self-assembly array to the cell self-assembly region. The present invention further provides a manufacturing method of a cell self-assembly array chip to solve the conventional problems which is difficult to culture living cells and not easy to apply optical observation.

### CROSS-REFERENCE

This application claims the priority of Taiwan Patent Application No. 101128953, filed on Aug. 10, 2012 in the Taiwan Intellectual Property Office (TIPO), the disclosure of which is incorporated herein in their entirety by reference. This invention is partly disclosed in a Symposium on Engineering Medicine and Biology Application (SEMBA) poster entitled “Rapid Cell-Assembly Chip for the Formation of High-Density Monolayer Cells Array” on Feb. 11, 2012 completed by Yu-Cheng Chang, Tsung-Ju Chen and Fan-Gang Tseng.

### FIELD OF THE INVENTION

The present invention relates to a cell chip and a manufacturing method thereof, and more particularly to a cell self-assembly array chip and a manufacturing thereof.

### BACKGROUND OF THE INVENTION

The observation and culture of specific cells are the basic and the most important part in bio-medical research. The method of cell observation according to the conventional technique is mainly to use a microscopy which involves the use of optical microscopy and the application of fluorescence microscopy. However, numerous and high-density cells are easily stacked with each other to form a multiple layer arrangement, and the stack of the multiple layer arrangement will have the problem of signal shadowing to generate wrong determination, so as to cause a mistake in detection. Therefore, to avoid the mistake in detection, it has to arrange the cells to be a single-layered array.

The single-layered cell array has the features of single-layer, high-density and numerous arrangements thereto. The method of arranging the cells to be a single-layered cell array according to the conventional technique comprises: chemical modification, dielectrophoresis and columnar and microwell structural constraints and so on, wherein the chemical modification is only used to attached type cells. Moreover, the design of working electrodes of dielectrophoresis is complicated to occupy a large amount of space for arrangement, so that it is not suitable for numerous cell arrangement, and the dielectrophoresis uses specific medium which is non-conventional cell culturing medium or buffer. Therefore, it may generate toxicity or damage to the cells. Furthermore, the cell array formed by the columnar structural constraint is used to be cell culture for single cell. Because of the design of columnar structural constraint needs extra space, so that a large amount of space will be wasted in the overall during detecting numerous cells. And, the microwell structural constraint limiting the cell array of the cells is used to examine and observe the response of single cell with drugs testing and rare cells detecting. However, because of the design of microwell structural constraint will occupy a large amount of space, so as to decrease the cell density in each unit of area.

For example, U.S. Pat. No. 6,548,263, entitled “Miniaturized cell array methods and apparatus for cell-based screening” is disclosed, wherein a selectable functional group, such as hydroxyl group, is used to modify a substrate, and can cause the cells attracting or rejecting the functional group to generate precipitation. Then, unattached cells are removed to form a single-layer cell array for be applied to tissue engineering. The formed cell array according to said method has low cell density, is not suitable for suspension cells, and will lose a large amount of cells. Furthermore, U.S. Pat. No. 7,358,079, entitled “Flow cell array and the utilization thereof for multianalyte determination” is disclosed, which uses an array of sample compartments each having an inlet and outlet to detect different cell samples. The disadvantage of said method is inconvenient to use and not suitable for high density cell array.

Additionally, for detecting a small amount of the cells, such as circulating tumor cells (CTCs), please refer to the publication of Nagrath, etc., entitled “Isolation of rare circulating tumor cells in cancer patients by microchip technology” (Nature 450, p. 1235-1239, 2007), wherein mentioned using EpCAM antibody to modify a plurality of microcolumns array on a microfluidic channel, and the CTCs are attached onto the microcolumns array through the specific binding between antibodies and antigens. Then, because of unattached cells are removed to leave the target cells, so that it achieves the purpose of scanning and detecting CTCs. The disadvantage of said method is difficult to capture all of the CTCs in a whole blood sample. When manufacturing the chips, it needs to use particular antibody to modify the microcolumns array, so that it decreases the universal application of the chips for various cells.

Therefore, the foregoing conventional cell array chips still have the following problems in practical usage, for example, it is not suitable for high density cell array, it is easy to lose a large amount of cells and due to antibody modification, it decreases the universal application of the chips for different cells. Furthermore, the foregoing conventional cell array chips and the purchasing/operating cost of the apparatus thereof are higher. As a result, it is necessary to provide a cell-assembly array chip and manufacturing method thereof, to solve the problems existing in the conventional techniques, as described above.

### SUMMARY OF THE INVENTION

A primary purpose of the present invention is to provide a cell self-assembly array chip, which has a cell self-assembly region and at least one drawing channel, and the structure of the chip can assist a fluid portion to vertically precipitate to the cell self-assembly region by gravity, and the liquid portion of cell suspension horizontally and radially pass through the drawing channel outward, wherein a radially outward pulling force is generated by the evaporation of the liquid portion, so as to cause that the cells are arranged onto the cell self-assembly region in a self-assembly manner. Therefore, the whole structure of the chip is easy and convenient to be assembled, and not necessary to use particular antibody to modify the surface of the substrate, so as to save the operation cost of the chip, suitable for a large amount of high-density cell detection and be universally applied to various cell samples. As a result, it can solve the technical problems of the conventional cell array chips which have too complicated design, waste the space in usage, limit the arranged number of cells and can not be universally applied to various cell samples.

A secondary purpose of the present invention is to provide a manufacturing method of a cell self-assembly array chip, which stacks a first substrate, spacer elements and a second substrate to be one-piece in turn, and after stacking, at least two fixing elements are used to clamp and fix the first substrate, spacer elements and the second substrate. Therefore, it actually simplifies the whole structure and the manufacturing process of the cell self-assembly array chip, so as to be advantageous to relatively decrease the manufacturing cost.

To achieve the above purpose, the present invention provides a cell self-assembly array chip which comprises

a first substrate formed with at least one first aperture;

a second substrate used as a base; and

a plurality of spacer elements annularly arranged on the second substrate at intervals, so as to define a cell self-assembly region and at least one drawing channel, wherein the cell self-assembly region is corresponding to the first aperture, and the first substrate, the spacer elements and the second substrate are stacked in turn;

wherein the cell self-assembly region is used to receive a cell suspension with cells, the cells of the cell suspension are vertically precipitated onto a surface of the cell self-assembly region by gravity, and the height of the drawing channel is smaller than the diameter of the cells, wherein a liquid portion of the cell suspension horizontally and radially passes through the drawing channel outward, and thus a radially outward drawing force is generated due to evaporation of the liquid portion of the cell suspension, so that the liquid portion of the cell suspension in the cell self-assembly region is reduced and thus the cells are arranged on the cell self-assembly region in a self-assembly manner to form a cell self-assembly array.

In one embodiment of the present invention, wherein further comprising: at least two fixing elements used to clamp and fix the first substrate, the spacer elements and the second substrate.

In one embodiment of the present invention, a top surface of the first substrate and an inner wall surface of the first aperture have an anti-adhesion modification layer.

In one embodiment of the present invention, the thickness of the second substrate is smaller than that of the first substrate.

In one embodiment of the present invention, the spacer elements are a plurality of curved photoresist strips equidistantly arranged on the second substrate.

The secondary purpose of the present invention is to provide a manufacturing method of a cell self-assembly array chip, comprising steps of:

providing a first substrate formed with a first aperture;

providing a second substrate used as a base;

annularly arranging a plurality of spacer elements on the second substrate at intervals, so as to define a cell self-assembly region and at least one drawing channel; and

stacking the first substrate, the spacer elements and the second substrate in turn;

wherein the cell self-assembly region is used to receive a cell suspension with cells, the cells of the cell suspension are vertically precipitated onto a surface of the cell self-assembly region by gravity, and the height of the drawing channel is smaller than the diameter of the cells, wherein a liquid portion of the cell suspension horizontally and radially passes through the drawing channel outward, and thus a radially outward drawing force is generated due to evaporation of the liquid portion of the cell suspension, so that the liquid portion of the cell suspension in the cell self-assembly region is reduced and thus the cells are arranged on the cell self-assembly region in a self-assembly manner to form a cell self-assembly array.

In one embodiment of the present invention, after the step of stacking the first substrate, the spacer elements and the second substrate in turn, further comprising a step of:

using at least two fixing elements to clamp and fix the first substrate, the spacer elements and the second substrate.

In one embodiment of the present invention, in the step of providing the first substrate, a top surface of the first substrate and an inner wall surface of the first aperture have an anti-adhesion layer.

In one embodiment of the present invention, the thickness of the second substrate is smaller than the thickness of the first substrate.

In one embodiment of the present invention, in the step of annularly arranging the spacer elements on the second substrate at intervals, comprising steps of:

coating a photoresist material onto the second substrate; and

exposing and developing the photoresist material to define a plurality of equidistantly arranged curved photoresist strips, so as to be the spacer elements.

### DESCRIPTION OF THE DRAWINGS

FIG. 1 is a top view of a first substrate of a cell self-assembly array chip according to a preferred embodiment of the present invention;

FIG. 2 is a top view of a second substrate of the cell self-assembly array chip according to the preferred embodiment of the present invention;

FIG. 3 is a cross-sectional view of the cell self-assembly array chip according to the preferred embodiment of the present invention;

FIG. 4 is another cross-sectional view of the cell self-assembly array chip according to the preferred embodiment of the present invention;

FIG. 5 is a partially enlarged view of the cell self-assembly array chip according to the preferred embodiment of the present invention; and

FIGS. 6A, 6B, 6C and 6D are schematic views of using the cell self-assembly array chip according to the preferred embodiment of the present invention.

### DETAILED DESCRIPTION OF THE PREFERRED EMBODIMENTS

The structure and the technical means adopted by the present invention to achieve the above and other objects can be best understood by referring to the following detailed description of the preferred embodiments and the accompanying drawings. Furthermore, directional terms described by the present invention, such as upper, lower, front, back, left, right, inner, outer, side, longitudinal/vertical, transverse/horizontal, and etc., are only directions by referring to the accompanying drawings, and thus the used directional terms are used to describe and understand the present invention, but the present invention is not limited thereto.

The present invention is provided a cell self-assembly array chip, which is designed to have a cell self-assembly region and at least one drawing channel in particular. The structure of the cell self-assembly array chip is able to assist a fluid portion to vertically precipitate by gravity, and then the fluid portion is horizontal and radially pass through the drawing channel outward, wherein a radially outward pulling force is generated by the evaporation effect of the liquid, so as to cause that the cells are arranged onto the cell self-assembly region in a self-assembly manner. Therefore, the whole structure of the chip is easy and convenient to be assembled, and not necessary to use particular antibody to modify the surface of the substrate, so as to save the operation cost of the chip, suitable for a large amount of high-density cell detection and be universally applied to various cell samples. As a result, it can solve the technical problems of the conventional cell array chips which have too complicated design, waste the space in usage, limit the arranged number of cells and can not be universally applied to various cell samples according to the conventional cell array chip.

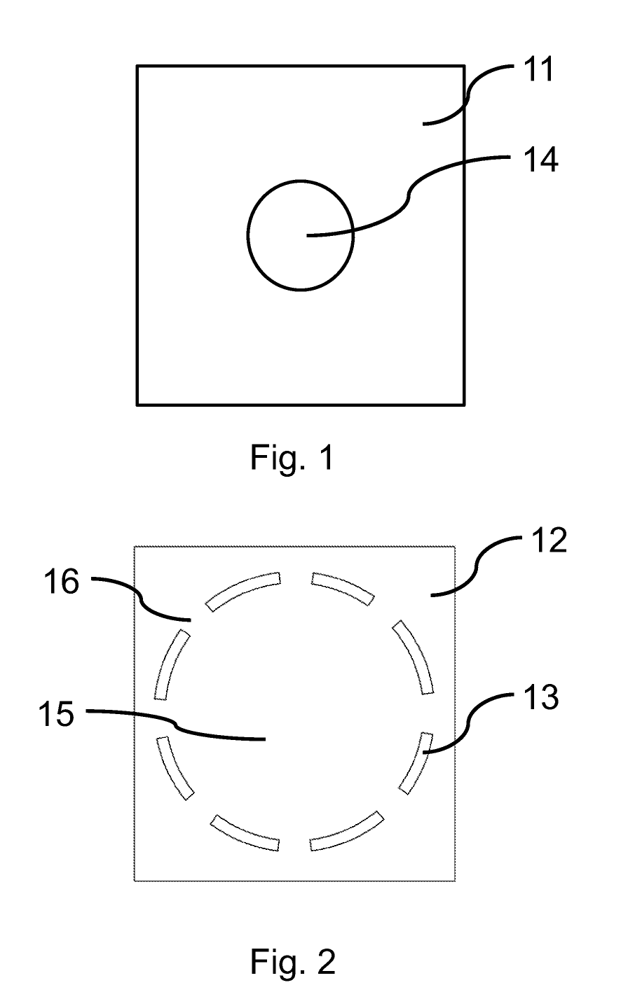

Referring now to FIGS. 1 and 2, a cell self-assembly array chip 10 according to a preferred embodiment of the present invention is illustrated. As shown, the cell self-assembly array chip 10 comprises: a first substrate 11, a second substrate 12 and a plurality of spacer elements 13. The first substrate 11 is formed with at least one first aperture 14 to be an injection portal for sample liquid, such as a cell suspension. The second substrate 12 is used as a base, and the thickness of the second substrate 13 is smaller than the thickness of the first substrate 11. The spacer elements 13 are arranged and fixed on the second substrate 12 at intervals to define a cell self-assembly region 15 and at least one drawing channel 16. The first substrate 11, the spacer elements 13 and the second substrate 12 are stacked to be one-piece in turn, wherein the materials of the first and the second substrates 11,12 can be glass or polydimethylsiloxane (PDMS), and the spacer elements 13 are preferably formed by exposing and developing a photoresist material (such as SU-8).

Referring back to FIGS. 1 and 2, the first substrate 11 is formed with at least one a first aperture 14, such as two, three, four or more, wherein the first aperture 14 is used to be an injection portal of a sample liquid, such as a cell suspension. One top surface of the first substrate 11 and an inner wall of the first aperture 14 preferably have an anti-adherent layer (not-shown), such as tridecafluoro-1,1,2,2-tetrahydrooctyl trichlorosilane (FOTS), to avoid the sample liquid from being overflowed or avoid the cells or microparticles of the sample liquid from adhering on the first substrate 11 to cause the loss of the cells and microparticles. Furthermore, the thickness of the second substrate 12 is preferably smaller than the thickness of the first substrate 11. However, if the thickness of the first or second substrates 11,12 is too thin, it will cause that mechanical strength thereof is insufficient; and if the thickness of the first or second substrates 11,12 is too thick, it will be disadvantageous for the observation of optical system, such as optical microscopy or fluorescence microscopy. Therefore, in the preferred embodiment of the present invention, the thickness of the first substrate 11 is preferably between 1 mm and 2 mm, and the thickness of the second substrate 12 is preferred to be within 0.5 mm and 1 mm.

Referring to FIG. 2 again, in the preferred embodiment of the present invention, a plurality of the spacer elements 13 are annularly arranged at interval and disposed on the second substrate 12 to define a cell self-assembly array region 15 and at least one drawing channel 16, wherein the spacer elements 13 are a plurality of curved photoresist strips equidistantly arranged on the second substrate 12. The cell self-assembly region 15 is a central region circled with a plurality of spacer elements 13, and the diameter of the cell self-assembly region 15 is greater than the diameter of the first aperture 14. Therefore, the number of the drawing channel 16 can be one or a plurality, such as two, three, four or more.

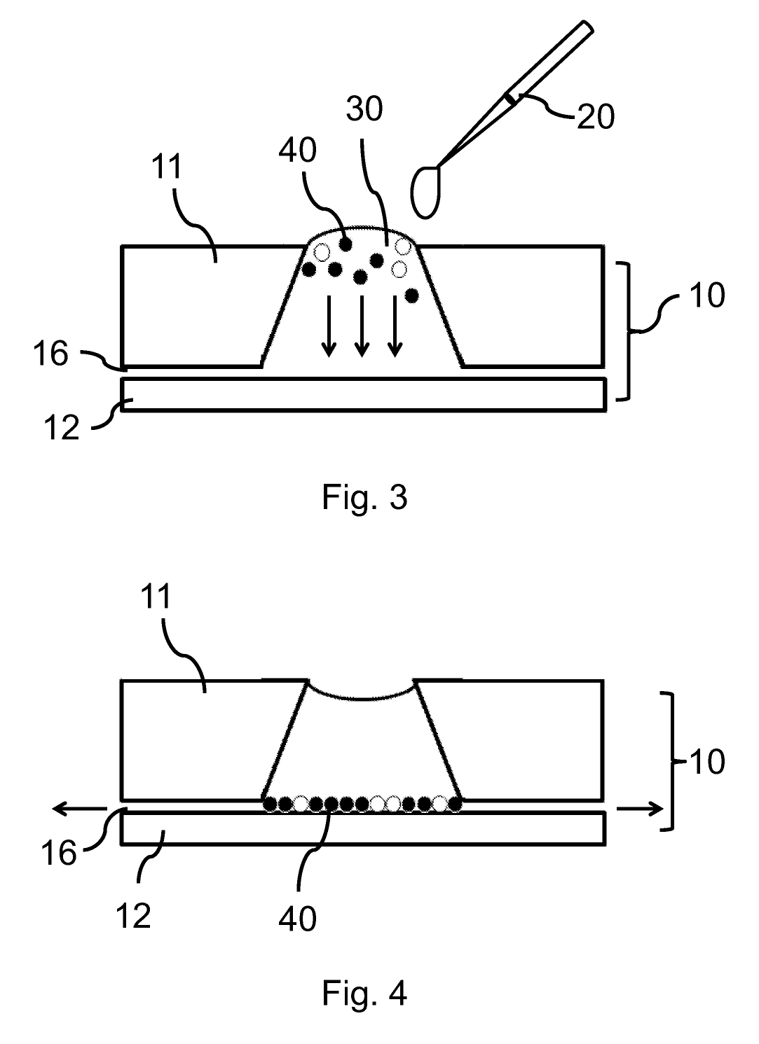

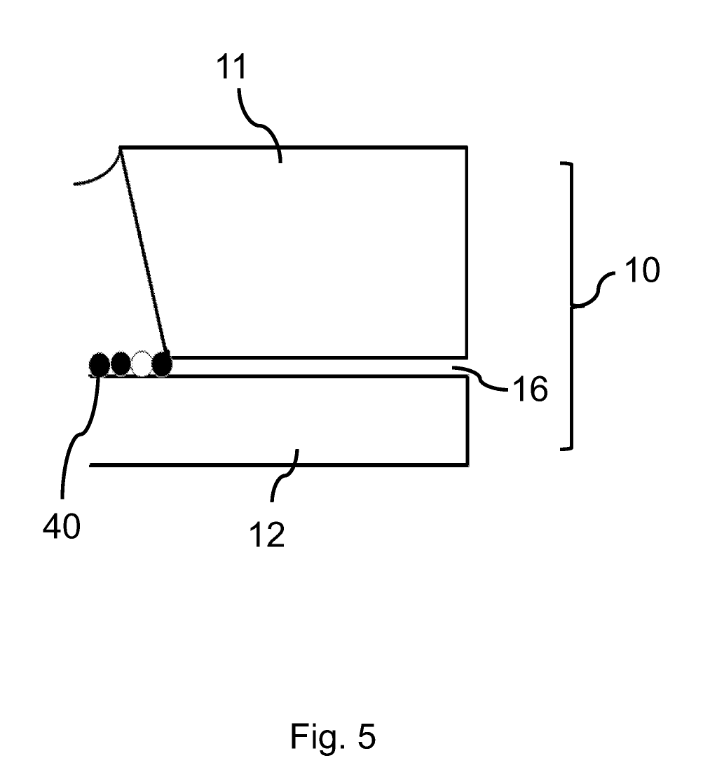

Referring to FIGS. 3 and 4, in the preferred embodiment of the present invention, when using the cell self-assembly array chip 10, the present invention provides a dropper 20 or a pipetman to take a sample liquid, such as a cell suspension, at first. The sample liquid is injected into the cell self-assembly array chip 10 from the first aperture 14, and precipitated on the cell self-assembly region 15 of the second substrate 12 by the weight of the liquid portion and cells due to the gravity, wherein the liquid portion of the suspension further passes through the drawing channel 16, referring to opposite drawing channels 16 as shown in FIG. 3. Therefore, because the evaporation effect of the liquid portion is generated outside the drawing channel 16 with the atmosphere, a radially outward pulling force is thus generated by the evaporation effect of the liquid portion, so as to cause that the liquid portion evaporates to decrease the liquid portion of the cell self-assembly region 15. Furthermore, the suspended particles 40 of the sample liquid, such as cells, are arranged onto the cell self-assembly region 15 in a self-assembly manner according to the decrease of the liquid portion and the precipitation of the cell due to the gravity. Additionally, it is worth to mention that the height (and the diameter) of the drawing channel 16 must be smaller than the average diameter of the cells, so that the drawing channel 16 is able to block the cells to pass through the drawing channel 16 for avoiding from losing the number of cells (as shown in FIG. 5).

As described above, the cell self-assembly array chip 10 of the preferred embodiment of the present invention, the structure of the chip is easy and convenient to be assembled, and not necessary to use particular antibody to modify the surface of the substrate, so as to save the operation cost of the chip, suitable for a large amount of high-density cell detection and be universally applied to various cell samples, so as to solve the technical problems of the conventional cell array chip which have too complicated design, waste the space in usage, limit the arranged number of cells and can not be universally applied to various cell samples.

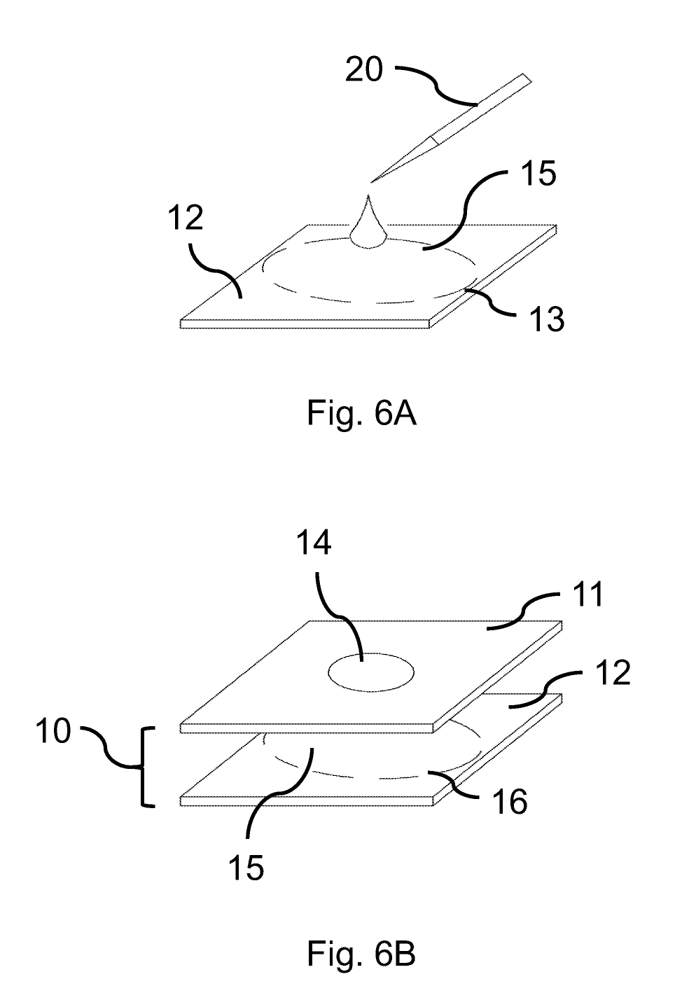

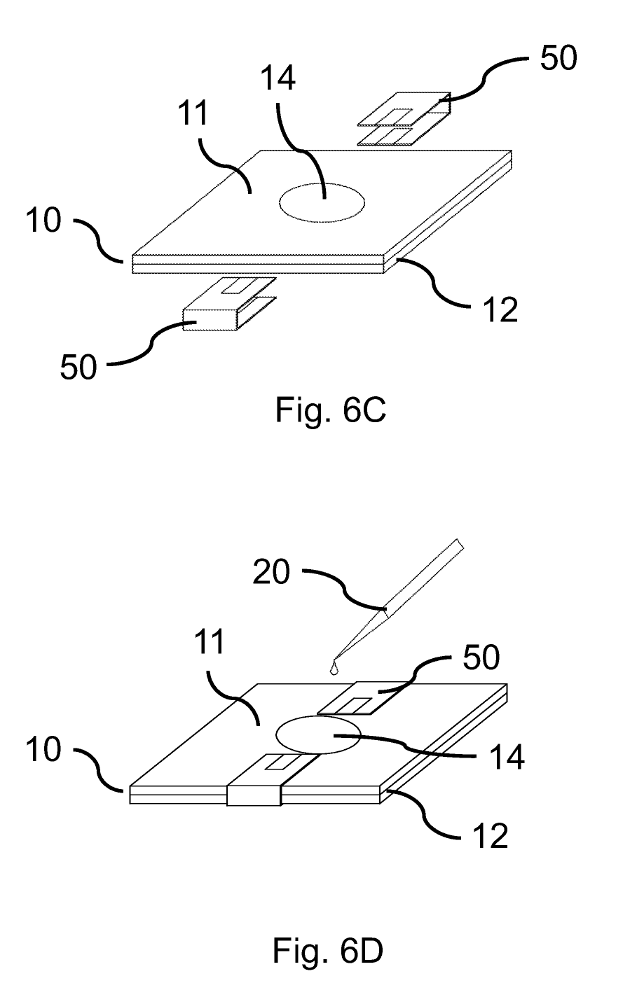

Moreover, referring to FIGS. 6A and 6B, the preferred embodiment of the present invention further provides a manufacturing method of a cell self-assembly array chip 10, which comprising steps of: providing a first substrate 11 formed with a first aperture 14; providing a second substrate 12 as a base; disposing a plurality of spacer elements 13 equidistantly arranged on the second substrate 12 to define a cell self-assembly region 15 and at least one drawing channel 16; stacking the first substrate 11, the spacer elements 13 and the second substrate 12 to be one-piece in turn, wherein the cell self-assembly region 15 is used to receive a cell suspension and the height of the drawing channel 16 is smaller than the diameter of the cells. Referring to

FIGS. 6C and 6D, in the embodiment of the present invention, if necessary, after the step of stacking the first substrate 11, the spacer elements 13 and the second substrate 12 in turn, the manufacturing method of the cell self-assembly array chip 10 further selectively comprises steps of: using at least two fixing elements 50 to clamp and fix the first substrate 11, the spacer elements 13 and the second substrate 12 for precisely controlling the gap between the first substrate 11 and the second substrate 12 (i.e. the height of the drawing channel 16).

For more details, referring to FIG. 6A again, in the preferred embodiment of the present invention, the step of arranging the spacer elements 13 equidistantly on the second substrate 12 comprises steps of: coating a photoresist material, such as SU-8, on the second substrate 12; and exposing and developing the photoresist material to define a plurality of equidistantly arranged curved photoresist strips, so as to be the spacer elements 13, wherein the material of the first and second substrates 11,12 can be glass or polydimethylsiloxane (PDMS), and the spacer elements 13 are a photoresist material, such as SU-8, but not limited thereto. In other embodiment of the present invention, the material of the spacer elements 13 can be an ultraviolet (UV) curing resin, thermo-curing resin or other curing resin, such as epoxy resin.

Additionally, referring to FIGS. 6A to 6D again, in the preferred embodiment of the present invention, an application method of the cell self-assembly array chip 10 of the present invention can be carried out with the foregoing manufacturing method at the same time, wherein the steps of the application method comprise: adding appropriate amount of colorless phosphate buffered saline (PBS) onto the second substrate 12, and the first substrate 11 is covered to remove the excess colorless PBS. Then, the fixing elements 50 are used to clamp and fix the first substrate 11, the spacer elements 13 and the second substrate 12 in turn, to finish an assembly of the cell self-assembly array chip 10. Next, a cell suspension containing appropriate amount of cells is dropped into the first aperture 14 of the first substrate 11, and the chip is kept and wait for 5 to 10 minutes on a horizontal place. Therefore, due to the gravity and the evaporation effect from the drawing channel 16, a high-density cell self-assembly array chip is formed in a self-assembly manner. After that, the chip is able to observe under an optical microscopy or florescent microscopy. Furthermore, it is worth to mention that, if it is necessary to detect or experiment the sample liquid, the sample liquid is pretreated to avoid the cell self-assembly array from being destroyed in additional process before using the cell self-assembly array chip 10.

As described above, the conventional cell self-assembly array chip is used to detect the sample liquid simultaneously, but the microstructures of the conventional cell self-assembly array chip waste too much space, so that the conventional chip is not suitable for forming a high-density cell array. Furthermore, because a large amount of the cells is lost in application process and the conventional cell self-assembly array chips are modified with antibody, it will decrease the flexibility of universal application for various cell types. Therefore, in contrast, the cell self-assembly array chip of the present invention has a simple structure and convenient to be assembled. Simultaneously, the manufacturing and application processes of the conventional cell self-assembly array chip are simplified, and the flexibility of various applications in chip assembled is increased. Consequently, it is effective to solve the problems of optical observation using the chip, and the cell self-assembly array chip can be used to culture living cells and suitable to various purposes in research and experiment, so that the applicable range of the cell self-assembly array chip of the present invention is thus extended.

The present invention has been described with a preferred embodiment thereof and it is understood that many changes and modifications to the described embodiment can be carried out without departing from the scope and the spirit of the invention that is intended to be limited only by the appended claims.

What is claimed is:

1. A cell self-assembly array chip, comprising:
a first substrate formed with at least one first aperture;
a second substrate used as a base; and
a plurality of spacer elements annularly arranged on the second substrate at intervals, so as to define a cell self-assembly region and at least one drawing channel, wherein the cell self-assembly region is corresponding to the first aperture, and the first substrate, the spacer elements and the second substrate are stacked in turn;
wherein the cell self-assembly region is used to receive a cell suspension with cells, the cells of the cell suspension are vertically precipitated onto a surface of the cell self-assembly region by gravity, and the height of the drawing channel is smaller than the diameter of the cells, wherein a liquid portion of the cell suspension horizontally and radially passes through the drawing channel outward, and thus a radially outward drawing force is generated due to evaporation of the liquid portion of the cell suspension, so that the liquid portion of the cell suspension in the cell self-assembly region is reduced and thus the cells are arranged on the cell self-assembly region in a self-assembly manner to form a cell self-assembly array.

2. The cell self-assembly array chip according to claim 1, further comprising:
at least two fixing elements used to clamp and fix the first substrate, the spacer elements and the second substrate.

3. The cell self-assembly array chip according to claim 1, wherein a top surface of the first substrate and an inner wall surface of the first aperture have an anti-adhesion modification layer.

4. The cell self-assembly array chip according to claim 1, wherein the thickness of the second substrate is smaller than that of the first substrate.

5. The cell self-assembly array chip according to claim 1, wherein the spacer elements are a plurality of curved photoresist strips equidistantly arranged on the second substrate.

6. A manufacturing method of a cell self-assembly array chip, comprising steps of:
providing a first substrate formed with a first aperture;
providing a second substrate used as a base;
annularly arranging a plurality of spacer elements on the second substrate at intervals, so as to define a cell self-assembly region and at least one drawing channel; and
stacking the first substrate, the spacer elements and the second substrate in turn;
wherein the cell self-assembly region is used to receive a cell suspension with cells, the cells of the cell suspension are vertically precipitated onto a surface of the cell self-assembly region by gravity, and the height of the drawing channel is smaller than the diameter of the cells, wherein a liquid portion of the cell suspension horizontally and radially passes through the drawing channel outward, and thus a radially outward drawing force is generated due to evaporation of the liquid portion of the cell suspension, so that the liquid portion of the cell suspension in the cell self-assembly region is reduced and thus the cells are arranged on the cell self-assembly region in a self-assembly manner to form a cell self-assembly array.

7. The manufacturing method of the cell self-assembly array chip according to claim 6, wherein after the step of stacking the first substrate, the spacer elements and the second substrate in turn, further comprising a step of:
using at least two fixing elements to clamp and fix the first substrate, the spacer elements and the second substrate.

8. The manufacturing method of the cell self-assembly array chip according to claim 6, wherein in the step of providing the first substrate, a top surface of the first substrate and an inner wall surface of the first aperture have an anti-adhesion layer.

9. The manufacturing method of the cell self-assembly array chip according to claim 6, wherein the thickness of the second substrate is smaller than the thickness of the first substrate.

10. The manufacturing method of the cell self-assembly array chip according to claim 6, wherein in the step of annularly arranging the spacer elements on the second substrate at intervals, comprising steps of:
coating a photoresist material onto the second substrate; and
exposing and developing the photoresist material to define a plurality of equidistantly arranged curved photoresist strips, so as to be the spacer elements.
